(19) United States
(12) Patent Application Publication
(10) Pub. No.: US 2021/0187174 A1
(43) Pub. Date: Jun. 24, 2021

(54) Dressing Interface With Integrated Fluid Conduit

(71) Applicant: KCI Licensing, Inc., San Antonio, TX (US)

(72) Inventor: Christopher Brian LOCKE, Bournemouth (GB)

(21) Appl. No.: 16/757,350

(22) PCT Filed: Oct. 4, 2018
(86) PCT No.: PCT/US2018/054302

§ 371 Date: Apr. 17, 2020

Related U.S. Application Data
(60) Provisional application No. 62/576,137, filed on Oct. 24, 2017.

(51) Int. Cl. A61M 1/00; A61F 13/00

(52) U.S. Cl. CPC A61M 1/0086; A61M 1/0084; A61F 13/00068; A61M 1/0088

(57) ABSTRACT

An apparatus for connecting a therapy device to a tissue site and a system and method for using the same is described. The apparatus includes a housing having a flange and a conduit interface. A sheath is coupled to the conduit interface. A conduit is inserted through the sheath and the conduit interface. The sheath forms a fluid seal around the conduit, and a fluid conductor is coupled to the conduit.

### RELATED APPLICATION

This application claims the benefit, under 35 U.S.C. § 119(e), of the filing of U.S. Provisional Patent Application No. 62/576,137, entitled “DRESSING INTERFACE WITH INTEGRATED FLUID CONDUIT,” filed Oct. 24, 2017, which is incorporated herein by reference for all purposes.

### TECHNICAL FIELD

The invention set forth in the appended claims relates generally to tissue treatment systems and more particularly, but without limitation, to supplemental drainage with negative-pressure and/or instillation therapy.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound can be washed out with a stream of liquid solution, or a cavity can be washed out using a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for providing drainage in a negative-pressure therapy environment are set forth in the appended claims. Illustrative embodiments are also provided to enable a person skilled in the art to make and use the claimed subject matter.

For example, in some embodiments, an apparatus for connecting a therapy device to a tissue site is described. The apparatus can include a housing having a flange and a conduit interface. A sheath may be coupled to the conduit interface. A conduit can be inserted through the sheath and the conduit interface. The sheath may form a fluid seal around the conduit, and a fluid conductor may be coupled to the conduit.

Additionally or alternatively, example methods may comprise connecting a therapy device to a tissue site having an undermined space. For example, in some embodiments a manifold can be applied to the tissue site, and a passage can be created through the manifold adjacent to the undermined space. A cover can be applied over the manifold, and a hole can be cut in the cover over the passage. A drain may be inserted through the hole and the passage into the undermined space. The drain can be fluidly coupled to a conduit inserted through a conduit interface of a housing. The housing can be moved down the conduit, extending a sheath coupled to the conduit interface and forming a fluid seal around the conduit. A flange of the housing can be attached to the cover, and the conduit can be coupled to the therapy device.

A system for connecting a therapy device to a tissue site is also described herein. The system can include a tissue interface configured to be disposed adjacent a tissue site, and a cover configured to be disposed over the tissue site to form a sealed therapeutic environment. A dressing interface can be configured to couple the therapy device to the sealed therapeutic environment. The dressing interface can include a body having a base and a drain port and a flexible coupling coupled to the drain port. A tube may have a first end configured to be coupled to the therapy device and a second end that may be inserted through the flexible coupling and the drain port. The flexible coupling may form a fluid seal around the tube. A drain can be coupled to the second end of the tube.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure therapy and/or instillation therapy with drainage in accordance with this specification;

FIG. 2 is a perspective view illustrating additional details that may be associated with an example embodiment of the therapy system of FIG. 1;

FIG. 3 is a sectional view taken along line 3-3 of FIG. 2, illustrating additional details of a dressing of the therapy system of FIG. 2;

FIGS. 4A and 4B are detail views of FIG. 3, illustrating additional details of the dressing during negative pressure therapy;

FIGS. 5A-5F are sectional views illustrating additional details that may be associated with the application and use of the dressing of FIG. 2;

FIG. 6 is a sectional view illustrating additional details that may be associated with another example embodiment of a dressing interface that can be used with the therapy system of FIG. 2;

FIGS. 7A and 7B are detail views illustrating additional details that may be associated with the dressing interface of FIG. 6 during negative-pressure therapy;

FIG. 8 is a perspective view illustrating additional details that may be associated with an example embodiment of a drain that may be used with the therapy system of FIG. 2;

FIG. 9 is a perspective view illustrating additional details that may be associated with an example embodiment of another drain that may be used with the therapy system of FIG. 2; and

FIG. 10 is a perspective view illustrating additional details that may be associated with an example embodiment of another drain that may be used with the therapy system of FIG. 2.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

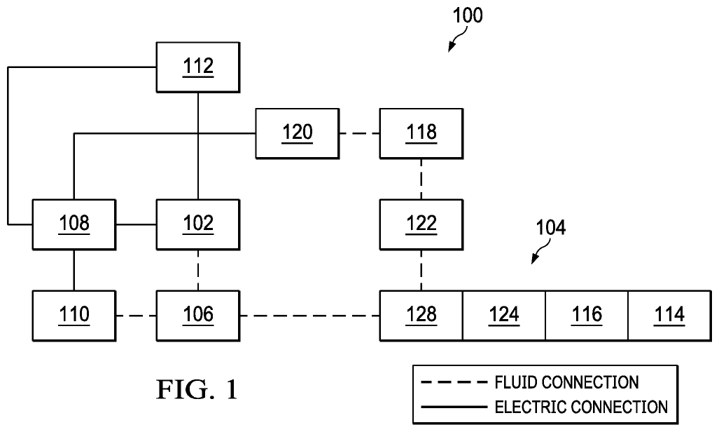

FIG. 1 is a simplified functional block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

Some tissue sites may include an undermined area or a tunnel. An undermined area can be a portion of a tissue site that extends under intact epidermal tissue peripheral to an opening at a surface of a tissue site. For example, the tissue site may be a wound having an opening through the epidermis. An undermined area may be a portion of the tissue site that extends laterally underneath intact epidermis. A tunnel may be a portion of the tissue site that extends further into the tissue. For example, a tissue site may extend through epidermal, dermal and subcutaneous tissue and having a concave shape. A tunnel can be a smaller opening formed in the concavity and extending an additional depth into the subcutaneous tissue.

The therapy system 100 may include a source or supply of negative pressure, such as a negative-pressure source 102, a dressing 104, a fluid container, such as a container 106, and a regulator or controller, such as a controller 108, for example. Additionally, the therapy system 100 may include sensors to measure operating parameters and provide feedback signals to the controller 108 indicative of the operating parameters. As illustrated in FIG. 1, for example, the therapy system 100 may include a pressure sensor 110, an electric sensor 112, or both, coupled to the controller 108. As illustrated in the example of FIG. 1, the dressing 104 may comprise or consist of a tissue interface 114, a cover 116, a drain 124, a dressing interface 128 or a combination of each in some embodiments.

The therapy system 100 may also include a source of instillation solution (e.g. saline). As illustrated in the example embodiment in FIG. 1, a solution source 118 may be fluidly coupled to the dressing 104. The solution source 118 may be fluidly coupled to a positive-pressure source such as a positive-pressure source 120, a negative-pressure source such as the negative-pressure source 102, or both in some embodiments. A regulator, such as an instillation regulator 122, may also be fluidly coupled to the solution source 118 and the dressing 104 to ensure proper dosage of instillation solution (e.g. saline) to a tissue site. For example, the instillation regulator 122 may comprise a piston that can be pneumatically actuated by the negative-pressure source 102 to draw instillation solution from the solution source during a negative-pressure interval and to instill the solution to a dressing during a venting interval. Additionally or alternatively, the controller 108 may be coupled to the negative-pressure source 102, the positive-pressure source 120, or both, to control dosage of instillation solution to a tissue site. In some embodiments, the instillation regulator 122 may also be fluidly coupled to the negative-pressure source 102 through the dressing 104, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the solution source 118, the controller 108 and other components into a therapy unit.

In general, components of the therapy system 100 may be coupled directly or indirectly. For example, the negative-pressure source 102 may be directly coupled to the container 106, and may be indirectly coupled to the dressing 104 through the container 106. Coupling may include fluid, mechanical, thermal, electrical, or chemical coupling (such as a chemical bond), or a combination of couplings in some contexts. For example, the negative-pressure source 102 may be electrically coupled to the controller 108, and may be fluidly coupled to one or more distribution components to provide a fluid path to a tissue site. In some embodiments, components may also be coupled by virtue of physical proximity, being integral to a single structure, or being formed from the same piece of material.

A distribution component is preferably detachable, and may be disposable, reusable, or recyclable. The dressing 104 and the container 106 are illustrative of distribution components. A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a fluid conductor, such as a tube, is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, a dressing interface, such as the dressing interface 128, may facilitate coupling a fluid conductor to the dressing 104. For example, such a dressing interface may be a SENSAT.R.A.C.™ Pad available from KCI of San Antonio, Tex.

A negative-pressure supply, such as the negative-pressure source 102, may be a reservoir of air at a negative pressure, or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 108, may be a microprocessor or computer programmed to operate one or more components of the therapy system 100, such as the negative-pressure source 102. In some embodiments, for example, the controller 108 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 114, for example. The controller 108 is also preferably configured to receive one or more input signals, such as a feedback signal, and is programmed to modify one or more operating parameters based on the input signals.

Sensors, such as the pressure sensor 110 or the electric sensor 112, are generally known in the art as an apparatus operable to detect or measure a physical phenomenon or property, and generally provide a signal indicative of the phenomenon or property that is detected or measured. For example, the pressure sensor 110 and the electric sensor 112 may be configured to measure one or more operating parameters of the therapy system 100. In some embodiments, the pressure sensor 110 may be a transducer configured to measure pressure in a pneumatic pathway and convert the measurement to a signal indicative of the pressure measured. For example, the pressure sensor 110 may be a piezoresistive strain gauge. The electric sensor 112 may optionally measure operating parameters of the negative-pressure source 102, such as the voltage or current, in some embodiments. Preferably, the signals from the pressure sensor 110 and the electric sensor 112 are suitable as an input signal to the controller 108, but some signal conditioning may be appropriate in some embodiments. For example, the signal may need to be filtered or amplified before it can be processed by the controller 108. Typically, the signal is an electrical signal, but may be represented in other forms, such as an optical signal.

The tissue interface 114 can generally be adapted to partially or fully contact a tissue site. The tissue interface 114 may take many forms, and may have many sizes, shapes, or thicknesses depending on a variety of factors, such as the type of treatment being implemented or the nature and size of a tissue site. For example, the size and shape of the tissue interface 114 may be adapted to the contours of deep and irregular shaped tissue sites. Moreover, any or all of the surfaces of the tissue interface 114 may have projections or an uneven, course, or jagged profile that can induce strains and stresses on a tissue site, which can promote granulation at the tissue site.

In some embodiments, the tissue interface 114 may be a manifold. A “manifold” in this context generally includes any substance or structure providing a plurality of pathways adapted to collect or distribute fluid across a tissue site under pressure. For example, a manifold may be adapted to receive negative pressure from a source and distribute negative pressure through multiple apertures across a tissue site, which may have the effect of collecting fluid from across a tissue site and drawing the fluid toward the source. In some embodiments, the fluid path may be reversed or a secondary fluid path may be provided to facilitate delivering fluid such as from a source of instillation solution across a tissue site.

In some illustrative embodiments, the pathways of a manifold may be interconnected to improve distribution or collection of fluids across a tissue site. In some illustrative embodiments, a manifold may be a porous foam material having interconnected cells or pores. For example, cellular foam, open-cell foam, reticulated foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

The average pore size of foam may vary according to needs of a prescribed therapy. For example, the tissue interface 114 may be foam having pore sizes in a range of about 400-600 microns. The tensile strength of the tissue interface 114 may also vary according to needs of a prescribed therapy. For example, the tensile strength of foam may be increased for instillation of topical treatment solutions. In some examples, the tissue interface 114 may be reticulated polyurethane foam such as found in GRANUFOAM™ dressing or V.A.C. VERAFLO™ dressing, both available from KCI of San Antonio, Tex.

The tissue interface 114 may be either hydrophobic or hydrophilic. In an example in which the tissue interface 114 may be hydrophilic, the tissue interface 114 may also wick fluid away from a tissue site, while continuing to distribute negative pressure to the tissue site. The wicking properties of the tissue interface 114 may draw fluid away from a tissue site by capillary flow or other wicking mechanisms. An example of a hydrophilic foam is a polyvinyl alcohol, open-cell foam such as V.A.C. WHITEFOAM™ dressing available from KCI of San Antonio, Tex. Other hydrophilic foams may include those made from polyether. Other foams that may exhibit hydrophilic characteristics include hydrophobic foams that have been treated or coated to provide hydrophilicity.

The tissue interface 114 may further promote granulation at a tissue site when pressure within the sealed therapeutic environment is reduced. For example, any or all of the surfaces of the tissue interface 114 may have an uneven, coarse, or jagged profile that can induce microstrains and stresses at a tissue site if negative pressure is applied through the tissue interface 114.

In some embodiments, the tissue interface 114 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 114 may further serve as a scaffold for new cell-growth, or a scaffold material may be used in conjunction with the tissue interface 114 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 116 may provide a bacterial barrier and protection from physical trauma. The cover 116 may also be constructed from a material that can reduce evaporative losses and provide a fluid seal between two components or two environments, such as between a therapeutic environment and a local external environment. The cover 116 may be, for example, an elastomeric film or membrane that can provide a seal adequate to maintain a negative pressure at a tissue site for a given negative-pressure source. The cover 116 may have a high moisture-vapor transmission rate (MVTR) in some applications. For example, the MVTR may be at least about 300 g/m² per twenty-four hours in some embodiments. In some example embodiments, the cover 116 may be a polymer drape, such as a polyurethane film, that is permeable to water vapor but impermeable to liquid. Such drapes typically have a thickness in the range of about 25-50 microns, inclusive of an attachment device. For permeable materials, the permeability generally should be low enough that a desired negative pressure may be maintained.

An attachment device may be used to attach the cover 116 to an attachment surface, such as undamaged epidermis, a gasket, or another cover. The attachment device may take many forms. For example, an attachment device may be a medically-acceptable, pressure-sensitive adhesive configured to bond the cover 116 to epidermis around a tissue site. In some embodiments, some or all of the cover 116 may be coated with an adhesive, such as an acrylic adhesive, which may have a coating weight between about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 118 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

In operation, the tissue interface 114 may be placed within, over, on, or otherwise proximate to a tissue site. If the tissue site is a wound, for example, the tissue interface 114 may partially or completely fill the wound, or may be placed over the wound. The cover 116 may be placed over the tissue interface 114 and sealed to an attachment surface near the tissue site. For example, the cover 116 may be sealed to undamaged epidermis peripheral to a tissue site. Thus, the dressing 104 can provide a sealed therapeutic environment proximate to a tissue site, substantially isolated from the external environment, and the negative-pressure source 102 can reduce the pressure in the sealed therapeutic environment. Negative pressure applied across the tissue site through the tissue interface 114 in the sealed therapeutic environment can induce macrostrain and micro-strain in the tissue site, as well as remove exudates and other fluids from the tissue site, which can be collected in container 106.

Negative-pressure therapy can provide evacuation of fluids and exudates from a tissue site, including a tissue site having an undermined area or a tunnel, and instillation therapy can provide therapeutic fluids to a tissue site, including an undermined area or a tunnel. In addition to negative-pressure therapy and/or instillation therapy, supplemental fluid conductors can be used to drain or to supply additional fluids to an undermined area or a tunnel of a tissue site. A separate exit for a supplemental fluid conductor may pass between the cover and the periwound epidermis. An exit between the periwound epidermis and the cover may be difficult to fluidly seal or require supplemental devices to ensure a fluid seal between the periwound epidermis and the cover. Ultimately, a separate exit from the sealed therapeutic environment can create an avenue for leakage across a cover. Leakage can decrease the efficiency of a negative-pressure source or an instillation source, decreasing the effectiveness of therapy.

Use of a supplemental fluid conductor for drainage or supplemental fluids can lead to other undesirable outcomes. For example, a supplemental fluid conductor used for drainage, such as a Jackson-Pratt drain, having a separate exit from the sealed therapeutic environment does not use negative pressure to draw fluids and exudates from the sealed therapeutic environment, which can decrease drainage efficiency. If the supplemental fluid conductor is inserted into the undermined area or tunnel, a user may have difficulty appropriately sizing the fluid conductor. For example, the volume of the sealed therapeutic environment can change during a cycle of negative-pressure therapy. If the supplemental fluid conductor is too long, the fluid conductor can impinge an end of the undermined area or tunnel when the sealed therapeutic environment is at a minimum volume, causing additional tissue trauma. Alternatively, if the supplemental fluid conductor is too short, the fluid conductor can be completely removed from the undermined area or tunnel when the sealed therapeutic environment is at a maximum volume, preventing the fluid conductor from providing drainage or supply of fluids to the undermined area or the tunnel.

A fluid conductor affixed to a dressing interface may manifold negative pressure only to an undermined area or tunnel and not to a tissue interface, which can decrease the effectiveness of the negative-pressure therapy. Devices having a fluid conductor affixed to a dressing interface may also require specialized training to place and use the fluid conductor and the dressing interface.

The therapy system 100 can overcome these and other problems by providing a fluid conductor coupled to a dressing interface. The fluid conductor can provide drainage or fluids to an undermined area or a tunnel while also providing distribution of fluids to a tissue interface. The fluid conductor can also move relative to the dressing interface. For example, in some embodiments of the therapy system 100, the therapy system 100 includes the drain 124, fluidly coupled to the dressing interface 128. The drain 124 provides drainage through the dressing interface 128, while accommodating the application of therapies, such as negative-pressure therapy and instillation therapy.

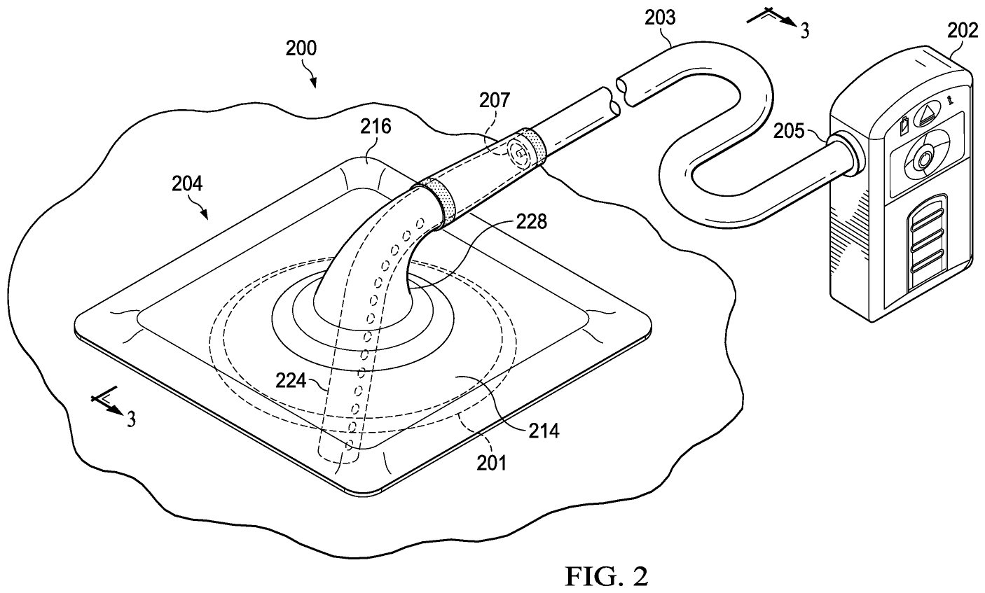

FIG. 2 is a perspective view of an example of a therapy system 200, illustrating additional details that may be associated with some embodiments. The therapy system 200 may be similar to and include elements of the therapy system 100 of FIG. 1. Similar components have similar numbers indexed to 200. The therapy system 200 can include a negative-pressure source 202 and a dressing 204 forming a sealed therapeutic environment over a tissue site 201. The negative-pressure source 202 may be fluidly coupled to the dressing 204 with a fluid conductor such as a conduit 203.

The negative-pressure source 202 may be similar to and operate as described above with respect to the negative-pressure source 102. The negative-pressure source 202 can include a container, similar to the container 106, a controller, similar to the controller 108, and one or more sensors, similar to the pressure sensor 110 and the electric sensor 112. The conduit 203 may have a first end 205 coupled to the negative-pressure source 202 and a second end 207. The conduit 203 may have one or more lumens configured to provide a fluid path between the dressing 204 and the negative-pressure source 202. For example, the conduit 203 may have a central lumen extending between the first end 205 and the second end 207. In other embodiments, the conduit 203 may have a central lumen or primary lumen and one or more peripheral lumens or ancillary lumens surrounding the central lumen.

The dressing 204 may be similar to and operate as described above with respect to the dressing 104. The dressing 204 can include a tissue interface 214, a cover 216, a drain 224, and a dressing interface 228. The tissue interface 214, the cover 216, the drain 224, and the dressing interface 228 may be similar to and operate as described above with respect to the tissue interface 114, the cover 116, the drain 124, and the dressing interface 128.

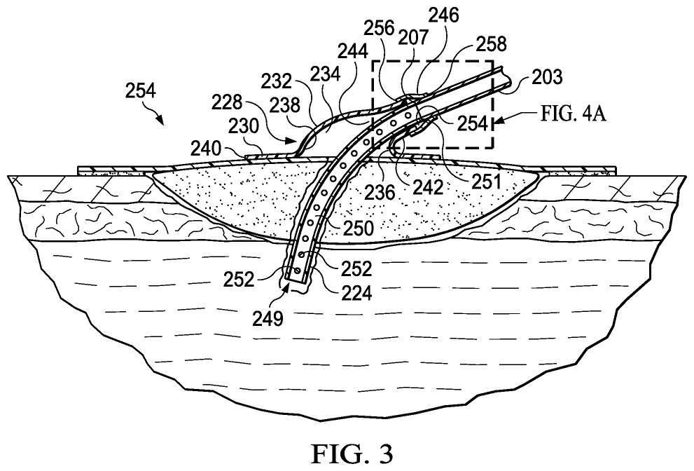

FIG. 3 is a sectional view taken along line 3-3 of FIG. 2, illustrating additional details of the dressing 204 of the therapy system 200 of FIG. 2. The dressing interface 228 may include a base, such as a flange 230 and a housing, such as a connector body 232. The connector body 232 may be a dome-shaped body forming a cavity 234 having a cavity aperture 236. In some embodiments, the cavity aperture 236 may have a circular shape. In other embodiments, the cavity aperture 236 may be ovoid or other shapes. The flange 230 may be an annular body having an inner diameter 238 and an outer diameter 240. The inner diameter 238 of the flange 230 may be coupled to the connector body 232 at the cavity aperture 236 and extend radially outward to the outer diameter 240. In some embodiments, the flange 230 and the cavity aperture 236 may be occupy a same horizontal plane, and the cavity aperture 236 and the inner diameter 238 of the flange 230 may be coincident.

A conduit interface, such as a drain port 242, may be coupled to the connector body 232. For example, the drain port 242 can be an elbow connector extending from the connector body 232. In the illustrated embodiment, the drain port 242 may be tubular body coupled to the connector body 232 proximate an apex of the connector body 232. The drain port may extend a distance radially outward and parallel to the flange 230. An end of the drain port 242 is spaced from an exterior surface of the connector body 232. In other embodiments, the drain port 242 may be coupled to an apex of the connector body 232 and extend away from the connector body perpendicular to the flange 230 so that an end of the drain port 242 is spaced from an exterior surface of the connector body 232. The drain port 242 may include a drain port lumen 244 fluidly coupled to the cavity 234 and configured to receive a fluid conductor, such as the drain 224 or the conduit 203. The drain port lumen 244 may have a radius between about 2.5 mm and about 3.5 mm. In some embodiments, the flange 230, the connector body 232, and the drain port 242 may be integrally formed into the dressing interface 228. For example, the dressing interface 228 may be mold, cast, or machined to include the flange 230, the connector body 232, and the drain port 242.

In some embodiments, the dressing interface 228 may include a drape ring or a drape pad. A drape ring may be a ring of adhesive material. A drape ring can be affixed to a surface of the flange 230 of the dressing interface 228 that is on an opposite side of the flange 230 from the drain port 242. A drape pad may be a ring of material similar to the cover 216. A drape pad can be affixed to a surface of the flange 230 of the dressing interface 228 that is on an opposite side of the flange 230 from the drain port 242. Both a drape ring and a drape pad may have an opening coincident with the cavity aperture 236. In some embodiments, the drape ring and a drape pad can couple the dressing interface 228 to the cover 216. In other embodiments, the dressing interface 228 can be affixed to the cover 216 using drape tape or other similar devices.

The drain 224 may have a distal end, such as a first end 249, and a proximal end, such as a second end 251. The drain 224 can also include at least one lumen 250, and a plurality of perforations 252. For example, the drain 224 may be a conduit having the at least one lumen 250 suitable for conveying fluid from the first end 249 of the drain 224 to the second end 251 of the drain 224. A length between the first end 249 and the second end 251 may be between about 50 mm and about 150 mm. The plurality of perforations 252 may penetrate a sidewall of the drain 224, providing fluid communication between the lumen 250 and a surrounding environment across the sidewall. In some embodiments, the plurality of perforations 252 may extend the length of the drain 224 from the first end 249 to the second end 251. In other embodiments, the plurality of perforations may extend a portion of the length of the drain 224 between the first end 249 and the second end 251. The plurality of perforations 252 may have a regularly repeating pitch both around a circumference of the drain 224 and the length of the drain 224. In other embodiments, the pitch of the plurality of perforations 252 may not be regularly repeating. The plurality of perforations 252 may each have a diameter sized to provide fluid flow to the lumen 250. For example, each perforation of the plurality of perforations 252 may have a diameter between about 1 mm and about 2 mm.

The first end 249 of the drain 224 may protrude from the cavity 234. For example, the first end 249 of the drain 224 extends from the cavity 234 through a plane occupied by the flange 230 and the cavity aperture 236. The second end 251 of the drain 224 may pass through the drain port 242. For example, the second end 251 of the drain 224 may pass through the drain port lumen 244 of the drain port 242. Preferably, the drain port lumen 244 of the drain port 242 may have a radius about 0.2 mm to about 0.5 mm larger than a radius of the exterior of the drain 224. For example, if the drain 224 has a radius of about 2 mm to about 3.5 mm, a diameter between about 4 mm and about 7 mm, the drain port lumen 244 may have a radius between about 2.5 mm and about 3.5 mm, a diameter between about 5 mm and about 7 mm. In some embodiments, the drain 224 may move through the drain port lumen 244 relative to the drain port 242. For example, the drain 224 and the drain port 242 may have a low relative coefficient of friction, permitting the drain 224 to slide through the drain port lumen 244 relative to the drain port 242.

The second end 251 of the drain 224 may be coupled to the second end 207 of the conduit 203, forming a solid bond between the drain 224 and the conduit 203. For example, the second end 251 of the drain 224 may be bonded, adhered, fused, welded, or otherwise joined to the second end 207 of the conduit 203. In an exemplary embodiment, the conduit 203 and the drain 224 may be formed of a polyurethane material, and the conduit 203 and the drain 224 may be coupled using an adhesive or joining component suitable for adhering polyurethane materials. In other embodiments, the drain 224 may be a portion of the conduit 203 having the plurality of perforations 252 formed therein. For example, the conduit 203 may have a portion that is perforated to form the plurality of perforations 252.

A flexible coupling, such as a sheath 246, can be coupled to the drain port 242. The sheath 246 can be a tube having an inner diameter 254 configured to receive an end of the drain port 242. In some embodiments, the sheath 246 may be formed from a polythene film, polyurethane film, or other soft flexible polymer film. The sheath 246 may have a wall thickness or film thickness between about 40 microns and about 70 microns. In other embodiments, the sheath 246 may have a wall thickness between about 70 microns and about 100 microns. A first end 256 of the sheath 246 may be coupled to the drain port 242. In some embodiments, the sheath 246 may be bonded to the drain port 242 using an adhesive or joining component. For example, the drain port 242 and the sheath 246 may be formed from a polyurethane material. The first end 256 of the sheath 246 may be placed around the drain port 242 and sealed to the drain port 242 using a suitable adhesive, such as an acrylic pressure sensitive adhesive. In other embodiments, a polyurethane, acrylic, silicone, or hydrogel adhesive having a thickness between about 20 gsm and about 50 gsm may be used. Preferably, the coupling process establishes a solid bond. A solid bond may be a coupling having a bond strength greater than the elastic limit of the materials being joined so that the material would fail prior to the bond between the materials failing.

The sheath 246 may have a second end 258 opposite the first end 256. The second end 258 of the sheath 246 may receive the conduit 203. For example, the conduit 203 having the drain 224 coupled to the second end 207 may be inserted into the second end 258 of the sheath 246 and through the drain port 242. The first end 249 of the drain 224 can protrude from the dressing interface 228 as described above. The second end 258 of the sheath 246 can be coupled to the conduit 203 so that the second end 258 of the sheath 246 moves with the conduit 203 in response to relative motion between the conduit 203 and the dressing interface 228. Preferably, the sheath 246 and the conduit 203 may be joined using a coupling method suitable for the materials of the respective components. For example, if the sheath 246 is formed from polyurethane and the conduit 203 is formed from polyurethane, an adhesive suitable for joining polyurethane materials may be used to adhere the sheath 246 to the conduit 203. In some embodiments, the second end 258 of the sheath 246 may be adhered to the conduit 203 using an acrylic pressure sensitive adhesive. In other embodiments, a polyurethane, acrylic, silicone, or hydrogel adhesive having a thickness between about 20 gsm and about 50 gsm may be used. Other coupling methods can be used to couple the sheath 246 and the conduit 203, including bonding, welding, friction coupling, or other suitable joining methods. In some embodiments, a portion of the conduit 203 may extend into the sheath 246, so that the second end 258 of the sheath 246 is spaced apart from the second end 207 of the conduit 203. In other embodiments, the sheath 246 can be coupled to the conduit 203 where the conduit 203 joins the drain 224. Preferably, the sheath 246 may be coupled to the drain port 242 and the conduit 203 so that the sheath 246 forms a fluid seal to both the drain port 242 and the conduit 203.

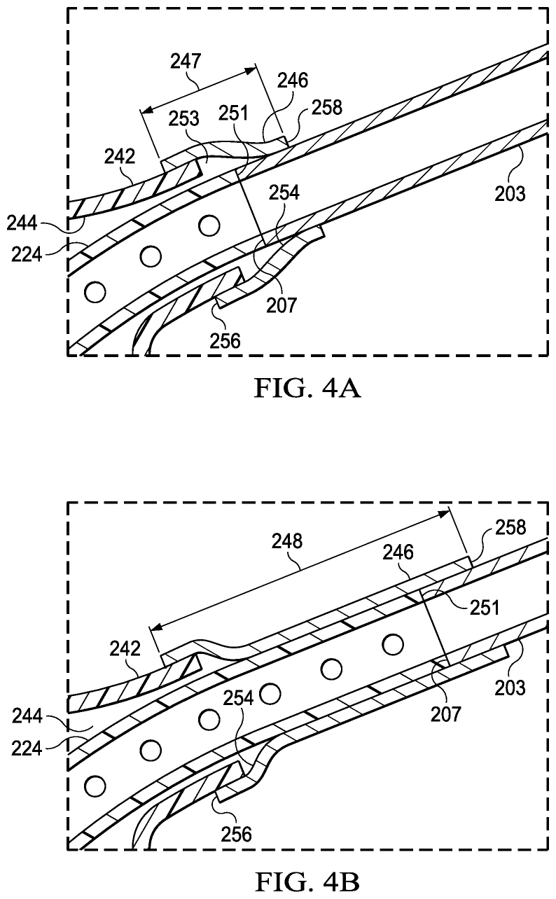

FIG. 4A and FIG. 4B are detail views of a portion of the dressing interface 228 of FIG. 3, illustrating additional details during negative-pressure therapy. In some embodiments, the sheath 246 may move between a first position and a second position. As shown in FIG. 4A, the sheath 246 may have a first length 247. The first length 247 of the sheath 246 may be between about 5 mm and about 10 mm. The sheath 246 and the drain 224 may form an annulus 253 between an exterior of the drain 224 and the inner diameter 254 of the sheath 246. As shown in FIG. 4B, the sheath 246 may have a second length 248. The second length 248 may be between about 50 mm and about 150 mm. The sheath 246 may have an elasticity permitting the sheath 246 to transition between the first length 247 and the second length 248. In some embodiments, the sheath 246 may transition between the first length 247 and the second length 248 by stretching. For example, the sheath 246 may be formed from a polyurethane material having an elasticity permitting an elongation to about three times the un-extended length of the sheath 246 without an elastic force urging retraction.

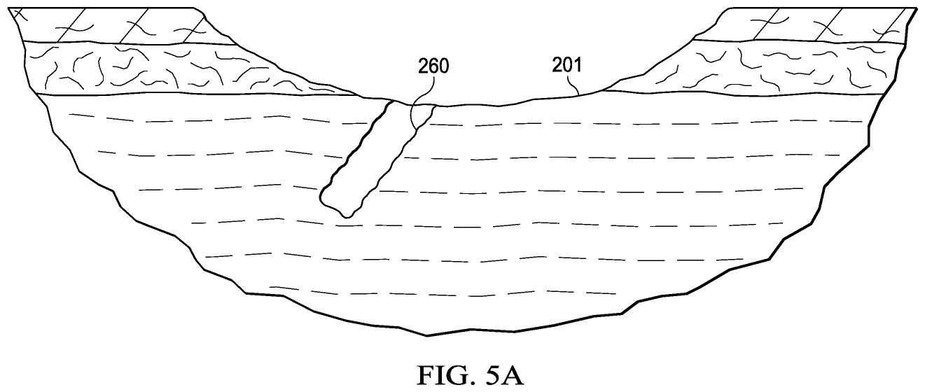

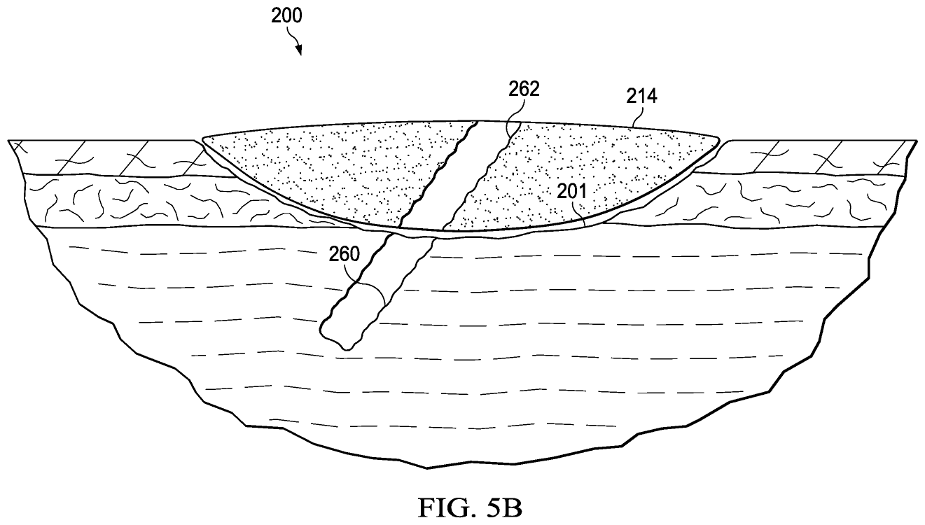

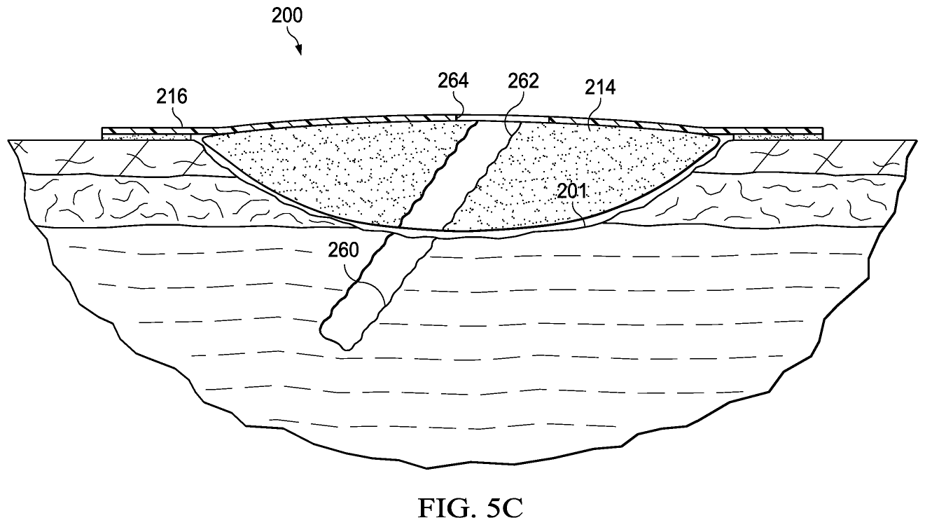

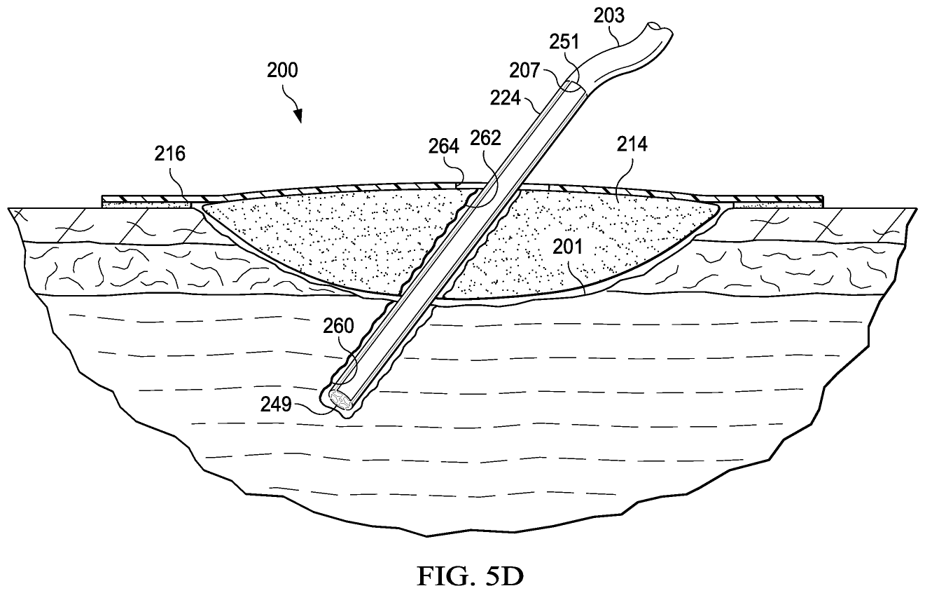

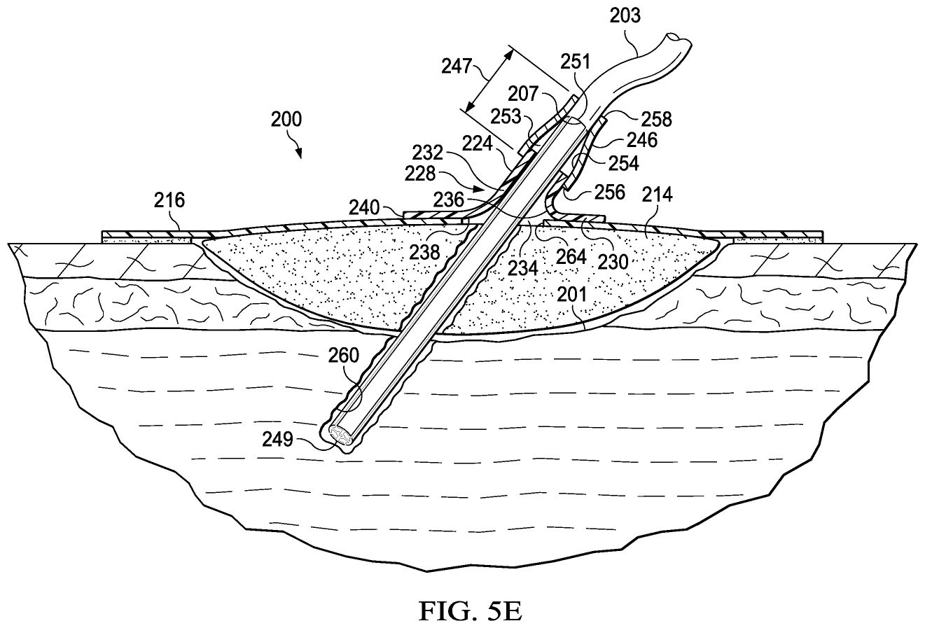

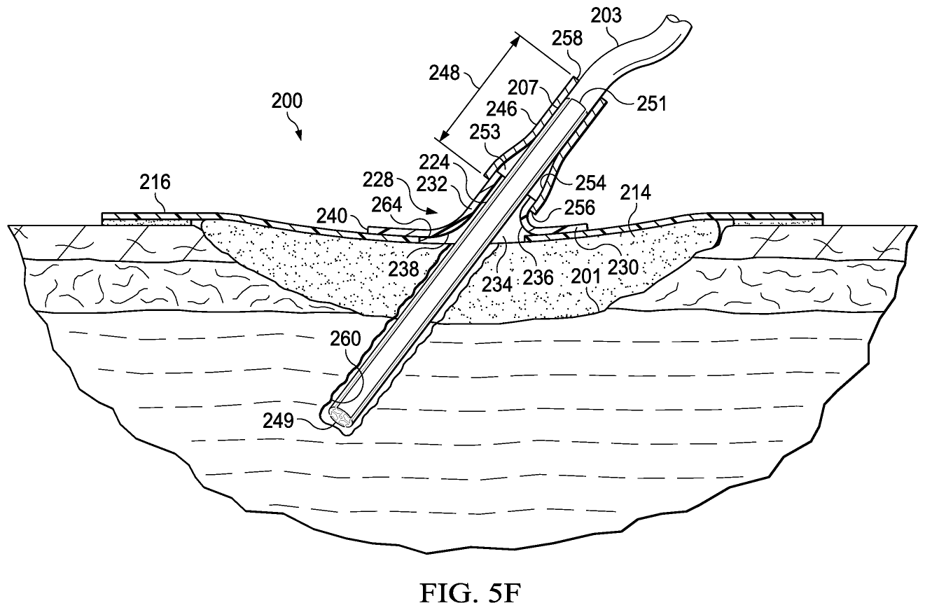

FIGS. 5A-5F are sectional views illustrating additional details that may be associated with application and use of the dressing 204 of FIG. 2. As shown in FIG. 5A, a tissue site 201 may have a tunnel 260. In some embodiments, a tissue site 201 may be evaluated to determine if the tissue site 201 includes the tunnel 260. The depth of the tunnel 260 can be evaluated to determine an approximate depth of the tunnel 260. In view of the depth of the tunnel 260, the drain 224 can be evaluated to determine if the drain 224 can be shortened, such as by cutting. The drain 224 may need to be shortened if the tunnel 260 is relatively shallow relative to the tissue site 201. Cutting may not be needed as the drain 224 and the dressing interface 228 may accommodate relative movement. As shown in FIG. 5B, the tissue interface 214 can be shaped and placed into the tissue site 201. Shaping the tissue interface 214 can include forming an opening 262 through the tissue interface 214. For example, a portion of the tissue interface 214 may be removed from a region of the tissue interface 214 above or adjacent to the tunnel 260.

As shown in FIG. 5C, a cover, such as the cover 216, can be placed over the tissue interface 214 and secured to epidermis surrounding the tissue site 201. An opening 264 can be cut in the cover 216. The opening 264 can be adjacent to the opening 262 formed in the tissue interface 214 over the tunnel 260. In some embodiments, the opening 264 may be larger than the opening 262 so that a portion of the surface of the tissue interface 214 is exposed through the cover 216. Preferably, the opening 264 may have a diameter approximately equal to a diameter of the cavity aperture 236. As shown in FIG. 5D, the drain 224 can be inserted through the opening 264 and the opening 262 and into the tunnel 260. In some embodiments, the second end 251 of the drain 224 may be flush with a surface of the tissue interface 214 exposed through the opening 264. Preferably, the second end 251 of the drain 224 may protrude from the opening 262 through the opening 264.

As shown in FIG. 5E, the dressing interface 228 can be secured to the cover 216. The dressing interface 228 may be coupled to the conduit 203 through the sheath 246. While the drain 224 is being inserted into the opening 262, the sheath 246 may have the first length 247. The flange 230 of the dressing interface 228 can be brought adjacent to the cover 216. In some embodiments, the cavity aperture 236 may be positioned coincident with the opening 264 in the cover 216. As the dressing interface 228 is brought adjacent to the cover 216, the sheath 246 may stretch from the first length 247. In some embodiments, the sheath 246 may stretch from the first length 247 to the second length 248. Preferably, the sheath 246 may stretch from the first length 247 to a length less than the second length 248.

Referring to FIG. 5F, the conduit 203 can be connected to the negative-pressure source 202, and the negative-pressure source 202 can be operated to draw fluid from the sealed therapeutic environment formed by the cover 216 and the dressing interface 228. As the negative-pressure source 202 draws fluid from the sealed therapeutic environment, atmospheric pressure can compress the tissue interface 214 and the cover 216 into the tissue site 201, decreasing a volume of the sealed therapeutic environment. As the dressing 204 compresses, the tissue interface 214 and the cover 216 may move toward the tunnel 260 of the tissue site 201; the flange 230 of the dressing interface 228 may move with the cover 216 through the coupling between the flange 230 and the cover 216. Movement of the flange 230 will similarly draw the connector body 232 and the drain port 242 toward the tunnel 260. The first end 249 of the drain 224 contacts a surface of the tunnel 260, preventing the drain 224 from moving further into the tunnel 260. In response, the conduit 203 will similarly not be drawn toward the tunnel 260. The sheath 246, coupling the conduit 203 to the drain port 242, can stretch, so that the optimal length of the drain 224 remains in the tunnel 260. In some embodiments, the sheath 246 stretches from the first length 247 to the second length 248 to accommodate the relative movement between the drain 224 and the dressing interface 228.

Fluid may be distributed through the tissue site 201 and the tunnel 260. For example, during negative-pressure therapy, the conduit 203 may draw fluid from the drain 224. In turn, the drain 224 may draw fluid from the tunnel 260 through the lumen 250. The drain 224 may also draw fluid form the cavity 234 through the plurality of perforations 252 exposed to the cavity 234 proximate the second end 251 of the drain 224. The fluid drawn from the cavity 234 can cause fluid to be drawn from the tissue interface 214 through the cavity aperture 236 and the opening 264 in the cover 216. Fluid may also be drawn from the tissue interface 214 through the plurality of perforations 252 of the drain 224 in portions of the drain 224 between the first end 249 and the second end 251 exposed to the tissue interface 214 through the opening 262. Similar fluid pathways may be formed during an instillation cycle; however, fluid may flow in the opposite direction.

In some embodiments, the annulus 253 may be formed between the exterior surface of the conduit 203, the exterior surface of the drain 224, and the inner diameter 254 of the sheath 246. As fluid is drawn from the sealed therapeutic environment, atmospheric pressure may urge the inner diameter 254 of the sheath 246 into contact with the drain 224, collapsing the annulus 253. The sheath 246 may collapse onto the exterior surface of the drain 224; however, the thickness of the film forming the sheath 246 can prevent the sheath 246 from being drawn into the plurality of perforations 252. The sheath 246 may cover the plurality of perforations 252, blocking fluid flow across the sidewall of the drain 224. Fluid flow can continue through the lumen 250 of the drain 224. If a therapeutic target pressure is reached in the sealed therapeutic environment, the sheath 246 may be fully collapsed around the drain 224, inhibiting further movement of the drain 224 relative to the dressing interface 228.

In some embodiments, the therapy system 200 may be used to provide instillation therapy. For example, the negative-pressure source 202 may be replaced with an instillation source. Fluids can be delivered to the tissue site 201 through the drain 224. In both an instillation therapy and a negative-pressure therapy environment, the sheath 246 may accommodate movement of the conduit 203 and the drain 224 relative to the dressing interface 228, while maintaining a fluid seal between the conduit 203 and the drain port 242. The ability to accommodate relative movement between the conduit 203 and the dressing interface 228 decreases instances of leaks from the sealed therapeutic environment due to patient movement.

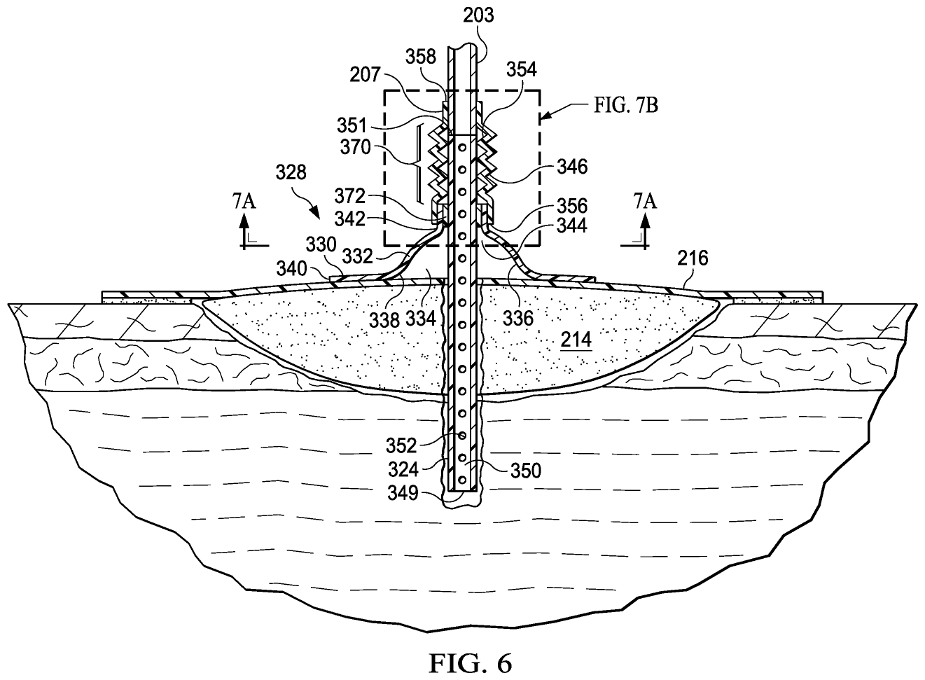

FIG. 6 is a sectional view, illustrating additional details of another drain 324 and dressing interface 328. The drain 324 and the dressing interface 328 may be similar to and operate as described above with respect to the drain 224 and the dressing interface 228. Similar components may have similar reference numbers indexed to 300. The dressing interface 328 may include a base, such as a flange 330 and a housing, such as a connector body 332, forming a cavity 334 and a cavity aperture 336. The flange 330 may be an annular body having an inner diameter 338 and an outer diameter 340. The inner diameter 338 of the flange 330 may be coupled to the connector body 332 at the cavity aperture 336 and extend radially outward to the outer diameter 340.

A drain port 342 may be coupled to the connector body 332. For example, the drain port 342 may be coupled to an apex of the connector body 332 and extend vertically and perpendicular to the flange 330 so that an end of the drain port 342 is spaced from an exterior surface of the connector body 332. The drain port 342 may include a drain port lumen 344 fluidly coupled to the cavity 334 and configured to receive a fluid conductor, such as the drain 324 or the conduit 203. In some embodiments, the flange 330, the connector body 332, and the drain port 342 may be integrally formed into the dressing interface 328. For example, the dressing interface 328 may be mold, cast, or machined to include the flange 330, the connector body 332, and the drain port 342. In some embodiments, the dressing interface 328 may include a drape ring or a drape pad. In some embodiments, the drape ring and a drape pad can couple the dressing interface 328 to the cover 216. In other embodiments, the dressing interface 328 can be affixed to the cover 216 using drape tape or other similar devices.

The drain 324 may have a distal end, such as a first end 349, and a proximal end, such as a second end 351. The drain 324 can also include at least one lumen 350, and a plurality of perforations 352. A length between the first end 349 and the second end 351 may be between about 50 mm and about 150 mm. The plurality of perforations 352 may penetrate a sidewall of the drain 324, providing fluid communication with the lumen 350 across the sidewall. In some embodiments, the plurality of perforations 352 may extend the length of the drain 324 from the first end 349 to the second end 351. The plurality of perforations 352 may each have a diameter sized to provide fluid flow to the lumen 350.

The first end 349 of the drain 324 may protrude from the cavity 334. For example, the first end 349 of the drain 324 extends from the cavity 334 through a plane occupied by the flange 330 and the cavity aperture 336. The second end 351 of the drain 324 may pass through the drain port 342. For example, the second end 351 of the drain 324 may pass through the drain port lumen 344 of the drain port 342. The second end 351 of the drain 324 may be coupled to the second end 207 of the conduit 203, forming a solid bond between the drain 324 and the conduit 203. For example, the second end 351 of the drain 324 may be bonded, adhered, fused, welded, or otherwise joined to the second end 207 of the conduit 203. Preferably, the drain port lumen 344 of the drain port 342 may have a diameter between about 5 mm and about 7 mm. Preferably, the radius of the drain port lumen 344 is about 0.2 mm to about 0.5 mm larger than a radius of the exterior of the drain 324. In some embodiments, the drain port 342 may include ribs or fins 372 extending radially inward from a surface of the drain port lumen 344. The fins 372 may be coupled to the surface of the drain port lumen 344. The fins 372 may extend a length of the drain port 342 from the cavity 334 to an end of the drain port 342. In some embodiments, the fins 372 may extend radially from a surface of the drain port 242 into the drain port lumen 344 between about 0.2 mm and about 0.5 mm.

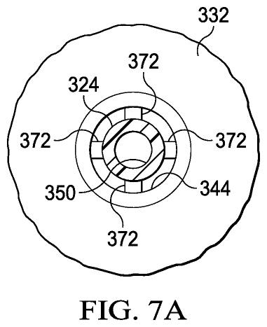

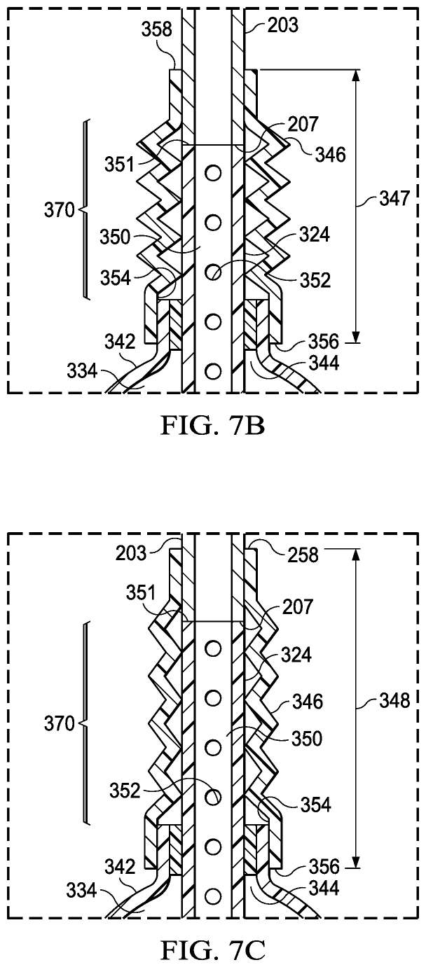

FIG. 7A is a sectional view taken along line 7A-7A of FIG. 6, illustrating additional details of the dressing interface 328. The fins 372 may be circumferentially spaced around the drain port lumen 344. In some embodiments, four fins 372 can be circumferentially spaced around the drain port lumen 344. In other embodiments, there may be more or fewer fins 372. For example, if the diameter of the drain port lumen 344 is increased, additional fins 372 may be used. Similarly, if the diameter of the drain port lumen 344 is decreased, fewer fins 372 may be used. In some embodiments, the drain 324 may move through the drain port lumen 344 relative to the drain port 342. For example, the drain 324 and the fins 372 may have a low relative coefficient of friction, permitting the drain 324 to slide through the drain port lumen 344 relative to the drain port 342.

FIG. 7B is a detail view illustrating additional details of the dressing interface 328 of FIG. 6. A flexible coupling, such as a sheath 346 having a bellows 370 can be coupled to the drain port 342 and the conduit 203. The sheath 346 can be a tube having an inner diameter 354 configured to receive an end of the drain port 342. In some embodiments, the sheath 346 may be formed from a polythene film, polyurethane film, or other soft flexible polymer film. The sheath 346 may have a wall thickness or film thickness between about 40 microns and about 70 microns. In other embodiments, the sheath 346 may have a wall thickness between about 70 microns and 100 microns. A first end 356 of the sheath 346 may be coupled to the drain port 342. In some embodiments, the sheath 346 may be bonded to the drain port 342 using an adhesive or joining component. For example, the drain port 342 and the sheath 346 may be formed from a polyurethane material. The first end 356 of the sheath 346 may be placed around the drain port 342 and sealed to the drain port 342 using a suitable adhesive, such as an acrylic pressure sensitive adhesive. In other embodiments, a polyurethane, acrylic, silicone, or hydrogel adhesive having a thickness between about 20 gsm and about 50 gsm may be used. Preferably, the coupling process establishes a solid bond.

The sheath 346 may have a second end 358 opposite the first end 356. The second end 358 of the sheath 346 may receive the conduit 203. For example, the conduit 203 having the drain 324 coupled to the second end 207 may be inserted into the second end 358 of the sheath 346 and through the drain port 342. The second end 358 of the sheath 346 can be coupled to the conduit 203 so that the second end 358 of the sheath 346 can move with the conduit 203. Preferably, the sheath 346 and the conduit 203 may be joined using a coupling method suitable for the materials of the respective components. For example, if the sheath 346 is formed from polyurethane and the conduit 203 is formed from polyurethane, an adhesive suitable for joining polyurethane materials may be used to adhere the sheath 346 to the conduit 203. In some embodiments, the second end 358 of the sheath 346 may be adhered to the conduit 203 using an acrylic pressure sensitive adhesive. Other coupling methods can be used to couple the sheath 346 and the conduit 203, including bonding, welding, friction coupling, or other suitable joining methods. In some embodiments, a portion of the conduit 203 may extend into the sheath 346, so that the second end 358 of the sheath 346 is spaced apart from the second end 207 of the conduit 203. In other embodiments, the sheath 346 can be coupled to the conduit 203 where the conduit 203 joins the drain 324. Preferably, the sheath 346 may be coupled to the drain port 342 and the conduit 203 so that the sheath 346 forms a fluid seal to both the drain port 342 and the conduit 203. In some embodiments, the sheath 346 may move between a first position and a second position facilitated by the bellows 370. The bellows 370 may be a portion of the sheath 346 having concertinaed sides, permitting the bellows 370 to expand and contract. As shown in FIG. 7A, the sheath 346 may have a length 347. The length 347 of the sheath 346 may be between about 5 mm and about 10 mm.

FIG. 7C is a detail view of FIG. 6, illustrating additional details of the dressing interface during negative-pressure therapy. Application of negative-pressure therapy to the sealed therapeutic environment formed by the cover 216 and the dressing interface 328 may move the dressing interface 328 toward a surface of the tissue site 201. In response, the bellows 370 may expand so that the sheath 346 may have a second length 348. The second length 348 may be between about 50 mm and about 150 mm.

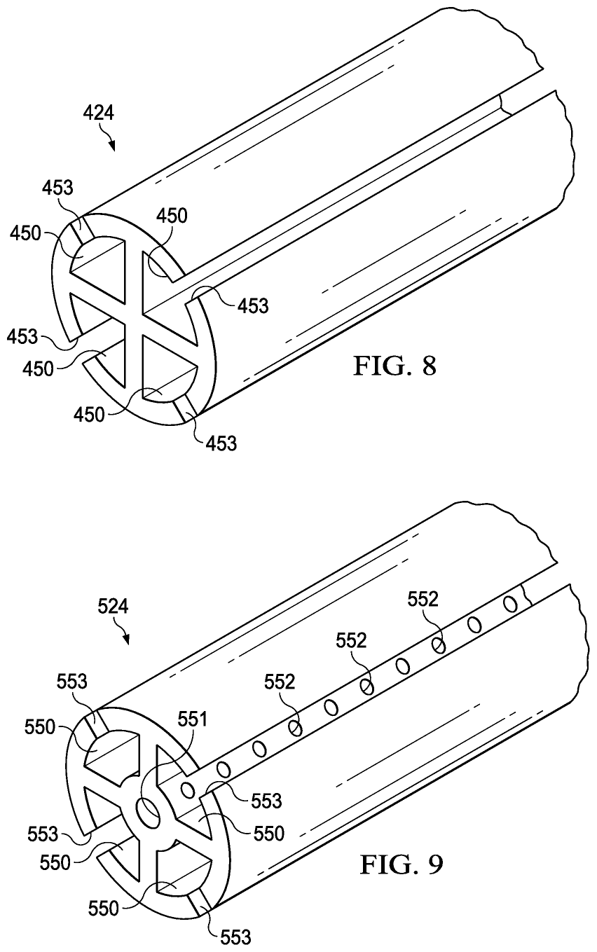

FIG. 8 is a perspective view illustrating additional details that may be associated with an example embodiment of a drain 424 that may be used with the therapy system of FIG. 2. The drain 424 may have a plurality of lumens 450 extending a length of the drain 424. In some embodiments, the drain 424 may include four lumens 450 circumferentially spaced around the drain 424. In other embodiments, the plurality of lumens 450 may be preferentially placed in a particular area of the drain 424, rather than circumferentially spaced. The drain 424 may also include a plurality of slits 453. Each slit of the plurality of slits 453 may extend a length of the drain 424. In some embodiments, the plurality of slits 453 may be circumferentially spaced around the drain 424. In other embodiments, the plurality of slits 453 may be preferentially placed in a particular area of the drain 424, rather than circumferentially spaced.

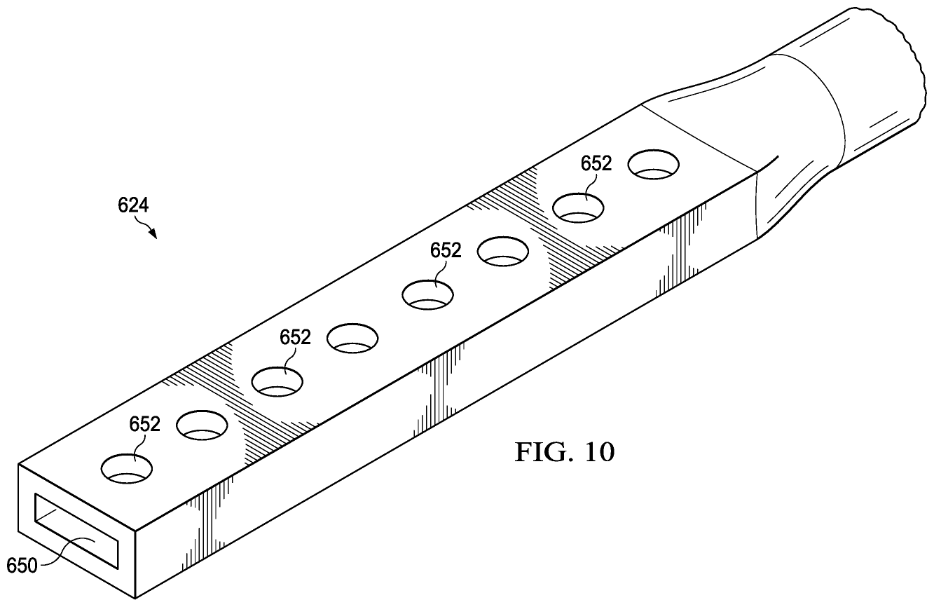

FIG. 9 is a perspective view illustrating additional details that may be associated with an example embodiment of a drain 524 that may be used with the therapy system 200 of FIG. 2. The drain 524 may have a plurality of lumens 550 extending a length of the drain 524. In some embodiments, the drain 524 may include four lumens 550 circumferentially spaced around the drain 524. In other embodiments, the plurality of lumens 550 may be preferentially placed in a particular area of the drain 524, rather than circumferentially spaced. The drain 524 can also include a central lumen 551. The central lumen 551 may be disposed along an axis of the drain 524 and extend a length of the drain 524. In some embodiments, the drain 524 may include a plurality of perforations 552. The plurality of perforations 552 may be disposed in a sidewall of the central lumen 551 and provide fluid communication between the central lumen 551 and the plurality of lumens 550. The drain 524 may also include a plurality of slits 553. Each slit of the plurality of slits 553 may extend a length of the drain 524. In some embodiments, the plurality of slits 553 may be circumferentially spaced around the drain 524. In other embodiments, the plurality of slits 553 may be preferentially placed in a particular area of the drain 524, rather than circumferentially spaced. In some embodiments, the plurality of slits 553 may be aligned with the plurality of perforations 552. In other embodiments, the plurality of slits 553 may be misaligned with the plurality of perforations 552.

In an embodiment, the plurality of perforations 552 may be removed. The central lumen 551 may be fluidly coupled to an instillation supply, and the plurality of lumens 550 may be fluidly coupled to the negative-pressure source 202. Fluid may be supplied to the tissue site 201 through the central lumen 551, and fluid may be drawn off and negative-pressure applied through the plurality of lumens 550.

FIG. 10 is a perspective view illustrating additional details that may be associated with an example embodiment of a drain 624 that may be used with the therapy system 200 of FIG. 2. The drain 624 may have at least one lumen 650 extending a length of the drain 624. In some embodiments, the drain 624 may include a plurality of perforations 652. The plurality of perforations 652 may be disposed in a sidewall of the at least one lumen 650 and provide fluid communication between the at least one lumen 650 and an area surrounding the drain 624. In some embodiments, the drain 624 may be disposed between the tissue site 201 and the tissue interface 214. The conduit 203 can then be coupled to an instillation source. In other embodiments, the drain 624 may be disposed between the tissue interface 214 and the cover 216. The conduit 203 can then be coupled to an instillation source. In still other embodiments, a portion of the tissue interface 214 can be disposed into the tissue site 201, the drain 624 can be disposed over the portion of the tissue interface 214, and another portion of the tissue interface 214 can be positioned over the drain 624. These arrangements can provide for preferential or maximum distribution of fluids via the perforated, hydrophilic foam media of the tissue interface 214. If negative pressure is also applied, effective wound washing may occur.

The systems, apparatuses, and methods described herein may provide significant advantages. For example, the drain and the dressing interface described herein provide a simplified application process. The dressing interface and the drain can also decrease the number of instances that the drain may have to be cut to appropriately fit an undermined area of a tissue site. The drain and the dressing interface can remove the need to provide separate exits from the sealed therapeutic environment. For example, a negative-pressure therapy connection and a drain connection can be provided through a single penetration of a cover, while providing seamless integration of a drain into the negative-pressure therapy application process. Furthermore, some embodiments described can remove fluids from undermined areas that may close or clog with components of a tissue interface. The drain and the dressing interface also manifold of negative pressure to the tissue interface as negative pressure may be distributed both at the top of the tissue interface and underneath the tissue interface through the drain. The described embodiments can also increase the duration of negative-pressure therapy. With use of some tissue interfaces, the tissue interface may become clogged with material from the tissue site, inhibiting distribution of negative pressure. The drain may also provide fluid removal from the sealed therapeutic environment as the tissue interface may become clogged with exudates and other material from the tissue site. The described embodiments provide an additional fluid path through the tissue interface, if the tissue interface becomes clogged.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 104, the container 112, or both may be eliminated or separated from other components for manufacture or sale. In other example configurations, the controller 108 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. An apparatus for connecting a therapy device to a tissue site, the apparatus comprising:
a housing comprising a flange and a conduit interface;
a sheath coupled to the conduit interface;
a conduit inserted through the sheath and the conduit interface, the sheath forming a fluid seal around the conduit; and
a fluid conductor coupled to the conduit.

2. The apparatus of claim 1, wherein the sheath comprises a flexible polymer film.

3. The apparatus of claim 1, wherein the sheath comprises a polythene film.

4. The apparatus of claim 1, wherein the sheath comprises a polyurethane film.

5. The apparatus of claim 1, wherein the sheath is a bellows.

6. The apparatus of claim 1, wherein the sheath has a wall thickness between about 30 microns and about 50 microns.

7. The apparatus of claim 1, wherein the sheath has a wall thickness greater than 50 microns.

8. The apparatus of claim 1, wherein the sheath has a wall thickness greater than 100 microns.

9. The apparatus of claim 1, wherein the sheath has a first length and a second length greater than the first length.

10. The apparatus of claim 9, wherein the sheath is configured to transition between the first length and the second length in response to movement of the housing.

11. The apparatus of claim 9, wherein the first length is between about 5 mm and about 10 mm.

12. The apparatus of claim 9, the second length is between about 50 mm and about 150 mm.

13. The apparatus of any preceding claim, wherein the conduit interface comprises an elbow connector.

14. The apparatus of any preceding claim, wherein the fluid conductor comprises a perforated conduit.

15. The apparatus of any preceding claim, wherein the fluid conductor comprises a proximal end coupled to the conduit, a distal end, and at least one channel between the proximal end and the distal end.

16. The apparatus of any preceding claim, wherein the conduit is a tube.

17. The apparatus of any preceding claim, wherein the conduit is a tube having a primary lumen and an ancillary lumen.

18. The apparatus of any preceding claim, wherein the conduit interface comprises a lumen having a surface and an axis and fins disposed on the surface parallel to the axis.

19. A method of connecting a therapy device to a tissue site having an undermined space, the method comprising:
applying a manifold to the tissue site;
creating a passage through the manifold adjacent to the undermined space;
applying a cover over the manifold;
cutting a hole in the cover over the passage;
inserting a drain through the hole and the passage into the undermined space, wherein the drain is fluidly coupled to a conduit inserted through a conduit interface of a housing;
moving the housing down the conduit, extending a sheath coupled to the conduit interface and forming a fluid seal around the conduit;
attaching a flange of the housing to the cover; and
coupling the conduit to the therapy device.

20. The method of claim 19, wherein inserting a drain comprises sizing the drain.

21. The method of claim 20, wherein sizing the drain comprises cutting a distal end of the drain.

22. The method of claim 19, wherein extending the sheath comprises stretching the sheath from a first length to a second length, the second length being longer than the first length.

23. The method of claim 22, wherein the first length is between about 5 mm to about 50 mm.

24. The method of claim 22, wherein the second length is between about 50 microns and about 150 microns.

25. The method of claim 19, wherein the sheath comprises a bellows and extending the sheath comprises expanding the bellows.

26. A system for connecting a therapy device to a tissue site, the system comprising:
a tissue interface configured to be disposed adjacent a tissue site;
a cover configured to be disposed over the tissue site to form a sealed therapeutic environment;
a dressing interface configured to couple the therapy device to the sealed therapeutic environment, the dressing interface comprising:
a body having a base and a drain port;
a flexible coupling coupled to the drain port;
a tube having a first end configured to be coupled to the therapy device and a second end inserted through the flexible coupling and the drain port, the flexible coupling forming a fluid seal around the tube; and
a drain coupled to the second end of the tube.

27. The system of claim 26, wherein the flexible coupling comprises a flexible polymer film.

28. The system of claim 26, wherein the flexible coupling comprises a polythene film.

29. The system of claim 26, wherein the flexible coupling comprises a polyurethane film.

30. The system of claim 26, wherein the flexible coupling is a bellows.

31. The system of claim 26, wherein the flexible coupling has a wall thickness between about 30 microns and about 50 microns.

32. The system of claim 26, wherein the flexible coupling has a wall thickness greater than 50 microns.

33. The system of claim 26, wherein the flexible coupling has a wall thickness greater than 100 microns.

34. The system of claim 26, wherein the flexible coupling has a first length and a second length greater than the first length.

35. The system of claim 34, wherein the flexible coupling is configured to transition between the first length and the second length in response to movement of the body.

36. The system of claim 35, wherein the first length is between about 5 mm and about 10 mm.

37. The system of claim 35, the second length is between about 50 mm and about 150 mm.

38. The system of any of claims 26-37, wherein the drain port comprises an elbow connector.

39. The system of any of claims 26-38, wherein the drain comprises a perforated tube.

40. The system of any of claims 26-39, wherein the drain comprises a proximal end coupled to the tube, a distal end, and at least one channel between the proximal end and the distal end.

41. The system of any of claims 26-40, wherein the tube is a tube having a primary lumen and an ancillary lumen.

42. The system of any of claims 26-41, wherein the drain port comprises a drain port lumen having a surface and fins disposed on the surface.

43. The system of any of claims 26-42, wherein the therapy device comprises a negative-pressure source.

44. The system of any of claims 26-42, wherein the therapy device comprises an instillation source.

45. The system of any of claims 26-42, wherein the therapy device comprises a combined negative-pressure and instillation therapy device.

46. The systems, apparatuses, and methods substantially as described herein.
